(19) United States
(12) Patent Application Publication
(10) Pub. No.: US 2009/0036909 A1
(43) Pub. Date: Feb. 5, 2009

(54) Femoral component holder

(76) Inventors: Lance D. Perry, Warsaw, IN (US); Jacy C. Hoeppner, Warsaw, IN (US)

Correspondence Address: HARNESS, DICKEY & PIERCE, P.L.C., P.O. BOX 828, BLOOMFIELD HILLS, MI, 48303, US

(21) Appl. No.: 11/097,929

(22) Filed: Apr. 1, 2005

(51) Int. Cl. A61B 17/08

(52) U.S. Cl. 606/157

(57) ABSTRACT

A holder for a femoral component. The holder includes a clamp adapted to engage an intercondylar feature of the femoral component and one of medial or lateral surfaces of the femoral component.

### INTRODUCTION

Various instruments are known for inserting, positioning, impacting or extracting femoral and tibial components in orthopedic knee procedures. As surgical knee procedures have been evolving to reduce the length of the procedure, trauma to associated tissues, and time for recovery, new instruments that can be used with standard or new procedures are desirable.

### SUMMARY

The present teachings provide a holder for a femoral component. The holder includes a clamp adapted to engage an intercondylar feature of the femoral component and one of the medial or lateral surfaces of the femoral component.

The present teachings provide a femoral component holder that includes a clamp having first and second members movable between open and closed clamp positions. One of the first and second members is adapted for engaging an intercondylar feature of the femoral component, and the other of the first and second members is adapted for engaging one of the lateral or medial surfaces of the femoral component.

The present teachings provide a method for implanting a femoral component. The method includes gripping an intercondylar feature of the femoral component, gripping one of the lateral or medial surfaces of the femoral component, clamping the femoral component between the intercondylar feature and the gripped lateral or medial surface, and positioning the femoral component on a prepared femur.

The present teachings also provide a holder for a femoral component that includes a clamp defining a medially offset axis and including medial and lateral extensions adapted for engaging the medial and lateral surfaces of the femoral component.

Further areas of applicability of the present invention will become apparent from the detailed description provided hereinafter. It should be understood that the detailed description and specific examples are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of a femoral component holder according to the present teachings, shown in a closed position holding a femoral component;

FIG. 2 is a side view of a femoral component holder according to the present teachings;

FIG. 3 is an exploded view of a femoral component holder according to the present teachings;

FIG. 4 is a partially exploded sectional view of a femoral component holder according to the present teachings;

FIG. 5A is an elevated side view of an arm for a femoral component holder according to the present teachings;

FIG. 5B is a front view of the arm of FIG. 5A;

FIG. 6A is an elevated side view of a leg for a femoral component holder according to the present teachings;

FIG. 6B is a front view of the leg of FIG. 6A;

FIGS. 7-9 are perspective views illustrating stages of holding, inserting and seating a femoral component using a femoral component holder according to the present teachings; and

FIG. 10 is a perspective environmental view of a femoral component holder according to the present teachings, shown in a closed position holding a femoral component.

### DETAILED DESCRIPTION

The following description is merely exemplary in nature and is in no way intended to limit the invention, its application, or uses. For example, although the present teachings are illustrated for applications with posterior-stabilized (PS) and cruciate-retaining (CR) femoral components in knee surgery, the present teachings can be used for other femoral components in knee surgery.

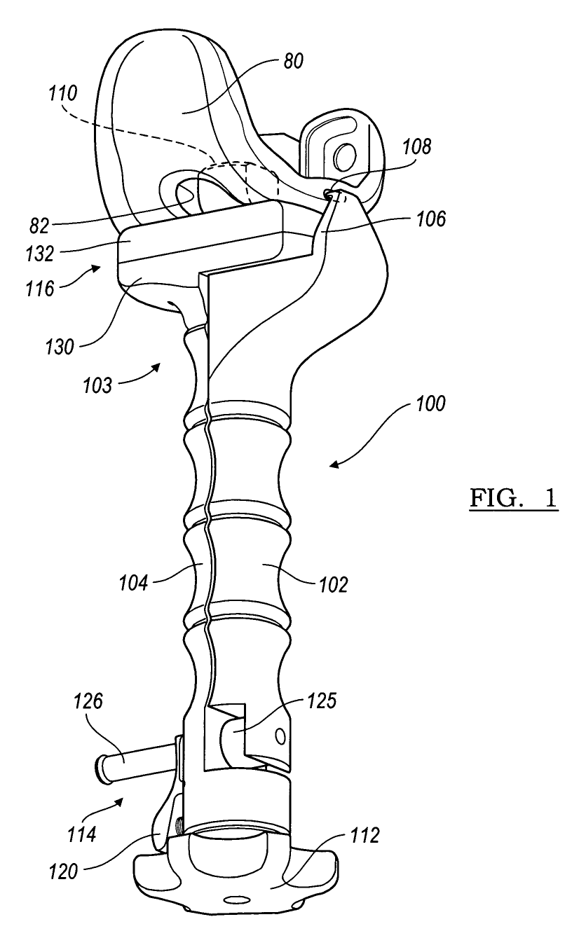

Referring to FIG. 1, an exemplary femoral component holder 100 according to the present teachings may include a clamp 103, a clamp actuator 114 and a locking device 112. The clamp 103 can include a leg 104 coupled to the locking device 112 and to a base 116. The clamp 103 can also include an arm 102 movably coupled to the leg 104. An intercondylar post 110 extending from the base 116 is adapted for gripping or generally engaging an intercondylar feature 82 of a femoral component 80. The intercondylar feature 82 can be an intercondylar recess or notch, an extension of the trochlear groove or other portion of the femoral component 80 intermediate between the lateral and medial sides of the femoral component 80, and is not limited to an intercondylar opening for a posterior stabilized knee prosthesis as shown for illustration purposes in FIG. 1.

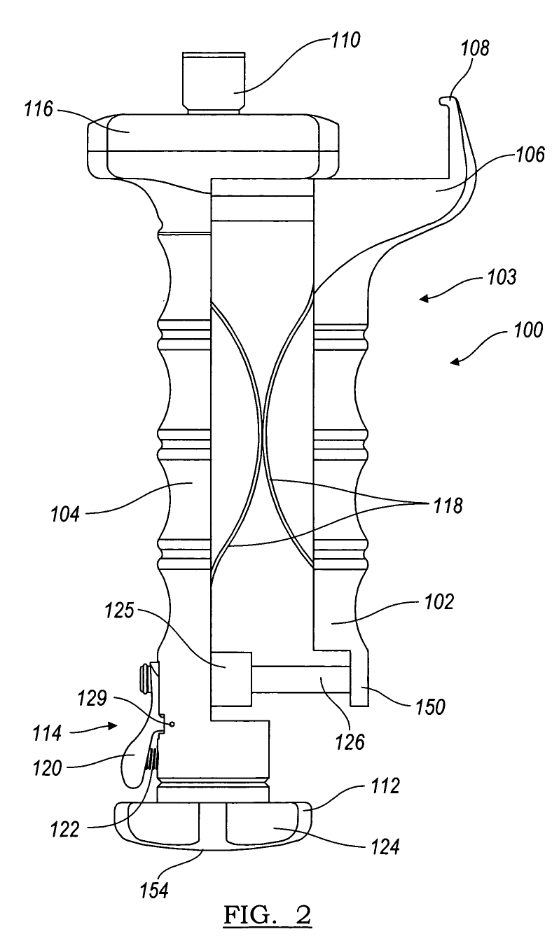

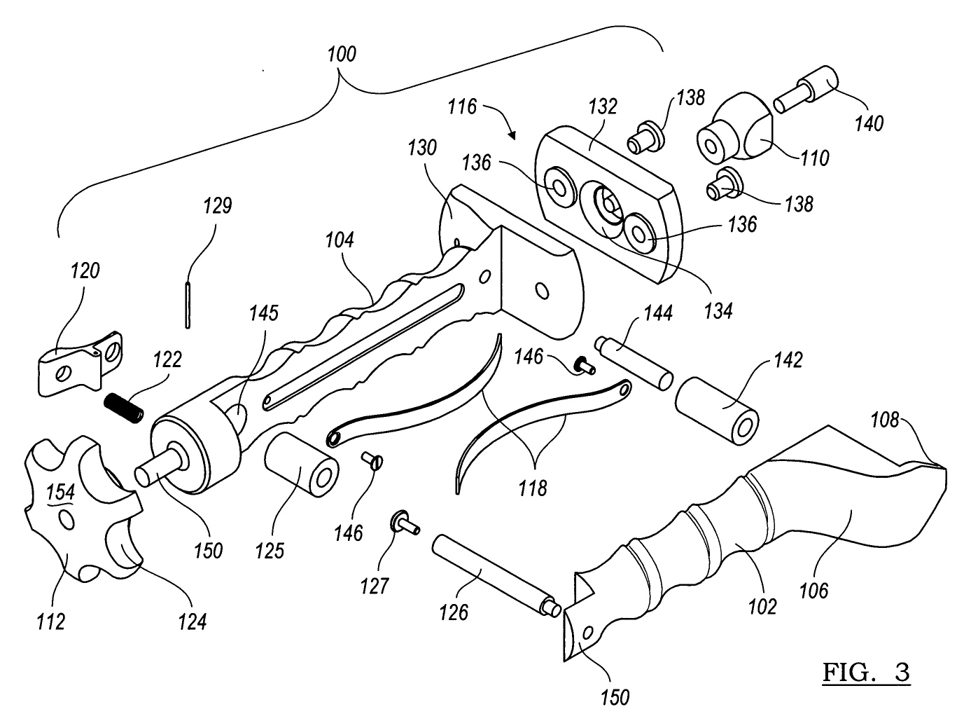

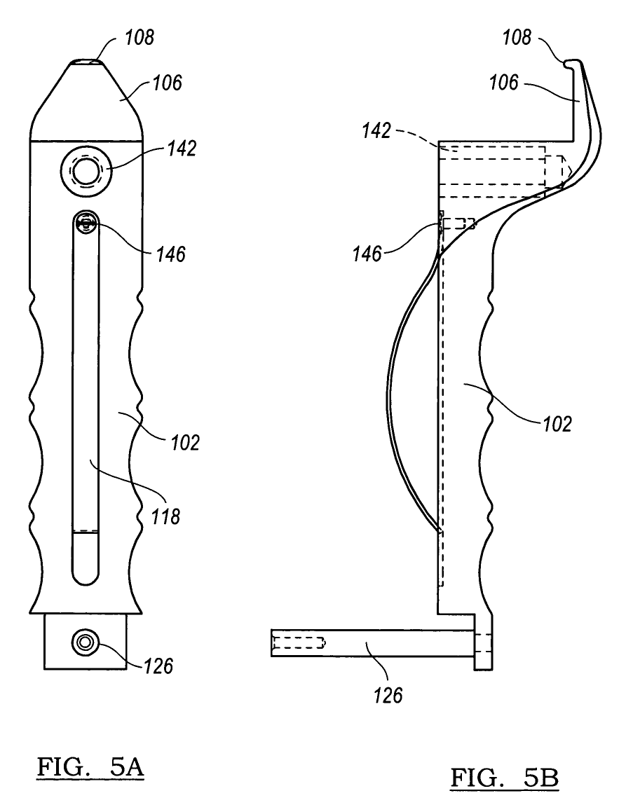

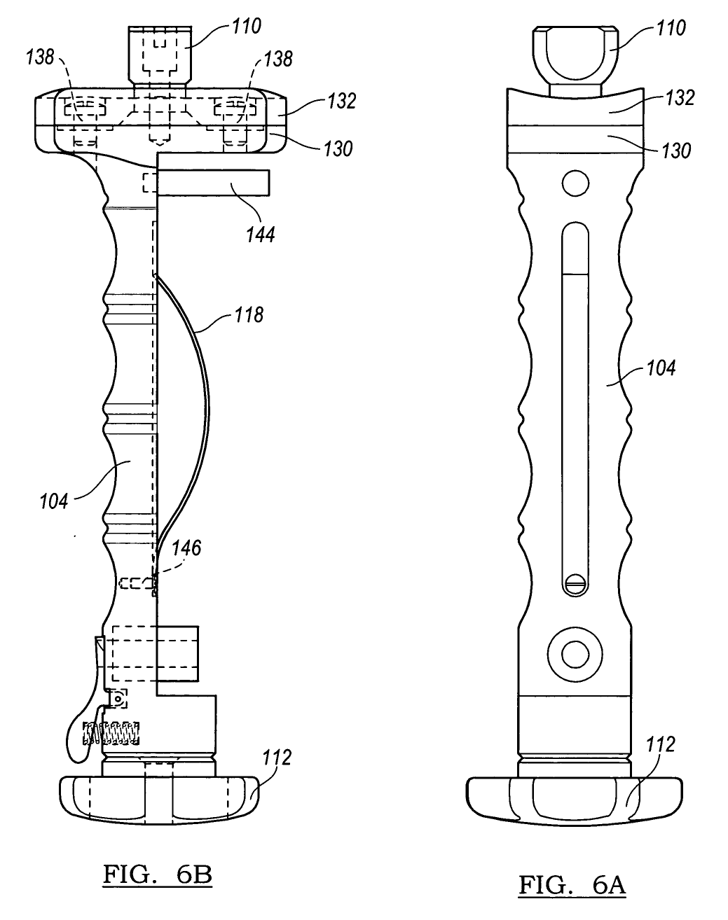

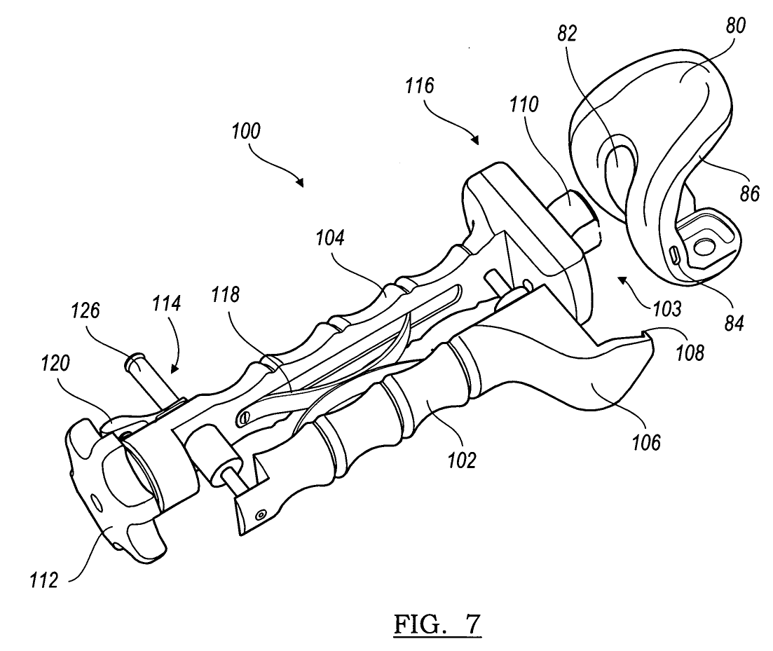

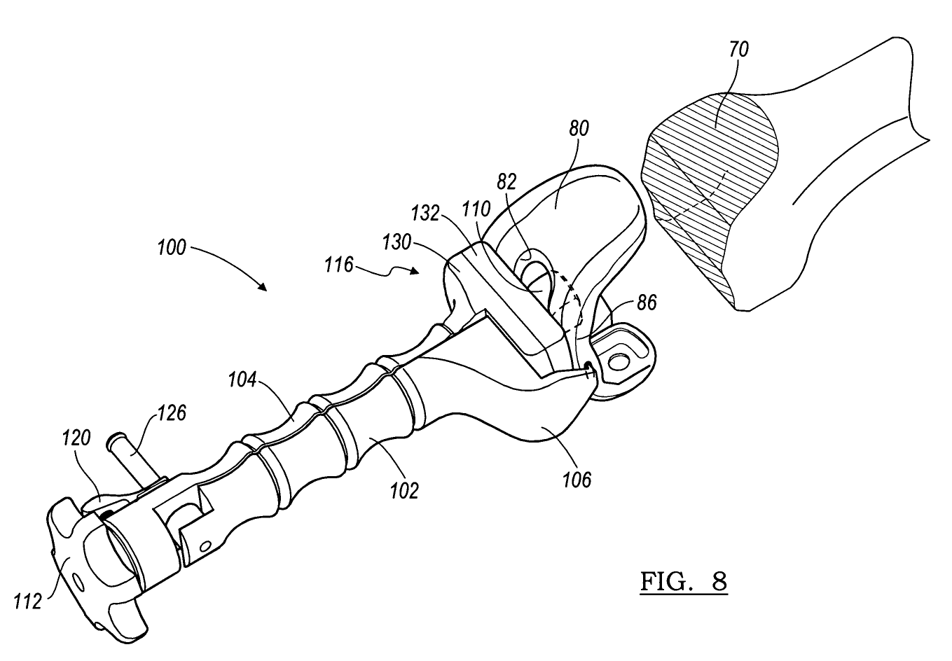

Referring generally to FIGS. 1-6 and, more particularly, to FIGS. 1-3, the holder 100 can include two leaf springs or other biasing element(s) 118 between the leg 104 and the arm 102. In the exemplary illustration of FIG. 3, two leaf springs 118 are opposingly coupled to the leg 104 and arm 102 with corresponding coupling devices 146, such as fasteners. The biasing element 118 biases the arm 102 from a closed clamp position illustrated in FIG. 1 to an open clamp position illustrated in FIG. 2, with the arm 102 moving substantially parallel to the leg 104.

An actuator 114 can be used to move the arm 102 between the closed and open clamp positions. The actuator 114, can include a handle or lever 120 that can pivot about a pin 129 coupled to the leg 104. The lever 120 can be coupled at one end to the arm 102 with a fastener that includes, for example, a shaft 126 passing through an opening 145 of the leg 104, and a head 127. The lever 120 is coupled at the other end to the leg 104 by means of a spring 122. Pushing the lever 120 against the biasing force of the spring 122 and the leaf springs 118 causes the shaft 126 to move through the opening 145, bringing an abutment surface 150 of the arm 102 against a tubular stop 125 through which the shaft 126 passes, thereby closing the clamp 103.

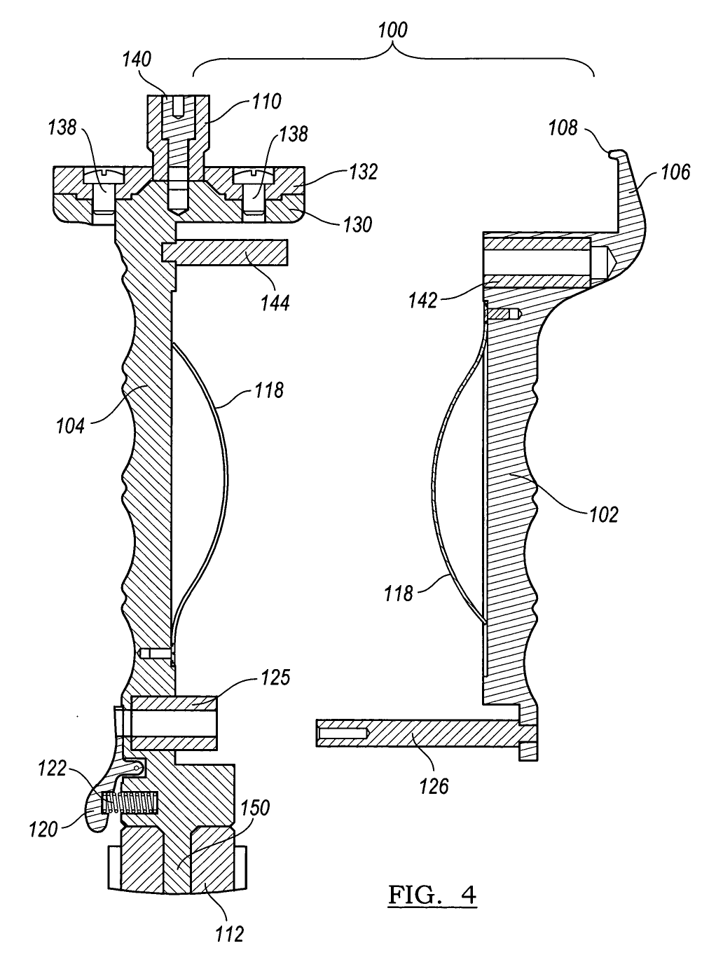

The arm 102 includes an extension 106 terminating in a hook or other engagement feature 108 adapted to engage a slot or other or recess 84 on one of the lateral or medial surfaces 86 of the femoral component 80. The arm 102 can also be coupled to the leg 104 with a base post 144 movably coupled to a bearing 142, as illustrated, for example, in FIG. 4.

The clamp 103 can be secured in the closed position by employing the locking device 112. The locking device 112, which can be, for example, knob-like, is rotatable coupled to a shaft 105 extending from the leg 104 and includes grooves or other engagement formations 124 adapted for capturing and locking the lever 120 in the closed position of the clamp 103 when rotated in one direction. Rotating the locking device 112 in the opposite direction, releases the lever 120. The locking device 112 can include an impaction surface 154 for applying impact force through the leg 104 to the base 116 for inserting or seating the femoral component 80.

Referring to FIG. 3, the base 116 can be modular and include, for example, a first plate 130 integrally or modularly coupled to the leg 104, and a second plate 132. The second plate 132 includes an opening 134 through which the intercondylar post 110 can be attached to the first plate 130 with a fastener or bolt 140. The second plate 132 can be a base pad for providing cushioning contact with the femoral component 80. The second plate 132 can be attached to the first plate 130 with recessed bolts or other fasteners 138 through holes 136 defined in the second plate 132.

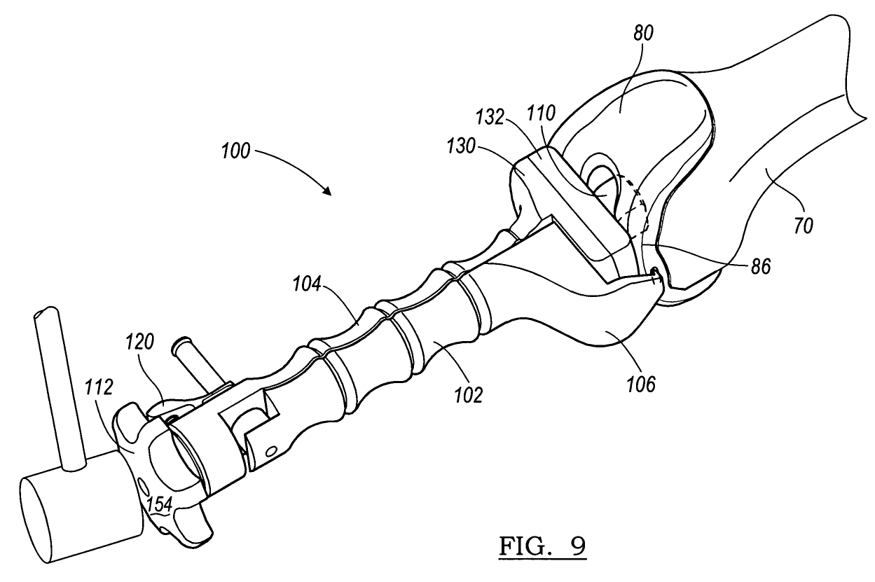

Referring to FIGS. 7-9, an exemplary method of using the holder 100 to implant the femoral component 80 is illustrated. Referring to FIG. 7, the lever 120 can be released by rotating the locking device 112, causing the clamp 103 to open. In the open clamp position, the intercondylar post 110 is brought into engagement with the intercondylar feature 82, while the hook 108 is brought opposite to the slot 84 of the femoral component 80.

Referring to FIG. 8, the lever 120 is pushed against the spring 122 to close the clamp 103, and the locking device 112 is rotated to lock the lever 120 in the closed clamp position with the holder 100 firmly engaging or clamping the femoral component 80 between the intercondylar post 110, and the extension 106. The femoral component 80 can also be engaged with the base 116. Referring to FIG. 9, the holder 100 is clamped on the femoral component 80 and is used to seat and impact the femoral component 80 on the prepared femur 70. The femoral component 80 can be fully seated on the femur by impacting the impaction surface 154 of the locking device 112.

As can be appreciated from the above description, the holder 100 according to the present teachings provides a relatively compact tool that combines the functions of gripping, positioning, inserting and impacting the femoral component, as desired, during a knee procedure. Further, at the discretion of the surgeon, the holder 100 can be used advantageously in minimally invasive procedures, as well as in standard surgical procedures. The holder 100 grips the femoral component 80 in only one of the lateral or medial surfaces 86, having a reduced width in comparison to known devices that grip both sides of the femoral component 80. Further, by gripping the femoral component only on one side, the holder 100 minimizes soft tissue damage during the procedure while providing versatility combined with a slim profile.

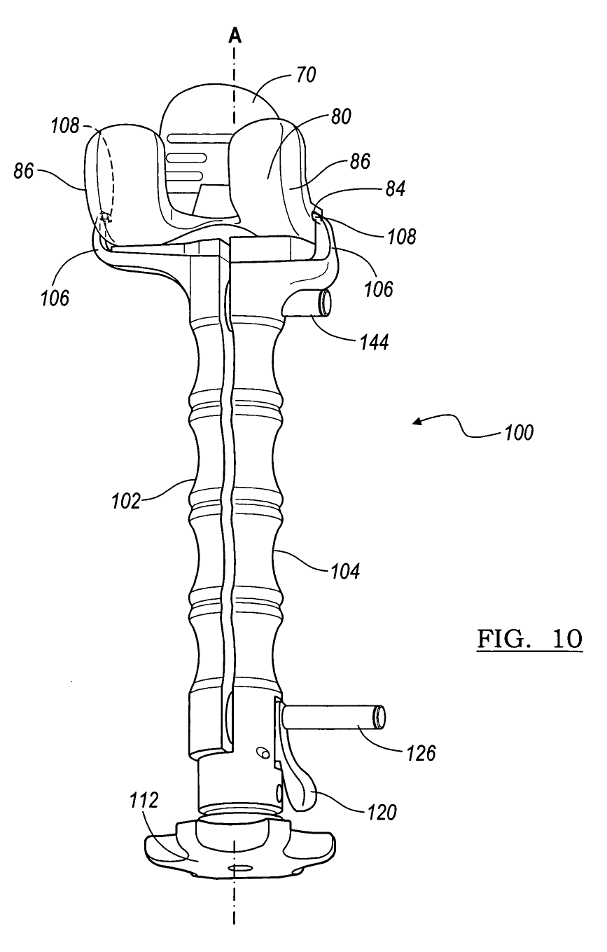

Referring to FIG. 10, an exemplary aspect of the femoral component holder 100 adapted for cruciate-retaining (CR) knee prostheses according to the present teachings is illustrated. In this aspect, the femoral component holder 100 does not include the intercondylar post 110 shown in FIG. 1, or it has been removed. Both the arm 102 and the leg 104 include corresponding extensions 106 terminating in hooks or fingers 108 for engaging in jaw-like fashion the medial and lateral surfaces 86 of the femoral component 80, as described above in connection with extension 106 associated with the arm 102. The holder 100 can have a longitudinal axis A, which can be medially offset relative to the extensions 106 and the medial and lateral surfaces 86 of the femoral component 80. Offsetting the axis A can facilitate the use of the holder 100, with typical or small incisions associated with minimally invasive surgery. In other aspects, the leg 104 can be integrally or modularly coupled with the associate extension 106, such that the leg 104 can be converted for engaging selectively a posterior stabilized prosthesis or a cruciate retaining prosthesis by adding/removing the intercondylar post 110 and removing/adding the leg extension 108.

The foregoing discussion discloses and describes merely exemplary arrangements of the present invention. One skilled in the art will readily recognize from such discussion, and from the accompanying drawings and claims, that various changes, modifications and variations can be made therein without departing from the spirit and scope of the invention as defined in the following claims.

1-14. (canceled)

15. A method for implanting a prosthetic femoral knee component, comprising:
providing a femoral knee component having an intercondylar notch between first and second condyles, an outer medial surface and a outer lateral surface, the intercondylar notch between the outer medial and lateral surfaces of the femoral component;
providing an inserter including a post and an engagement feature;
gripping the intercondylar notch of the femoral knee component with the post of the inserter;
gripping only one of the outer lateral or medial surfaces of the femoral knee component with the engagement feature of the inserter;
clamping only one of the first or second condyles of the femoral knee component between the intercondylar notch and the gripped outer lateral or medial surface between the post and the engagement feature of the inserter; and
positioning the femoral knee component on a prepared femur.

16. The method of claim 15, further comprising impacting the femoral knee component on the femur.

17. The method of claim 15, wherein clamping further comprises securing the femoral knee component in a clamped position.

18. The method of claim 17, further comprising releasing the femoral knee component from the clamped position.

19-28. (canceled)

29. A surgical apparatus comprising:
a prosthetic femoral knee component including an outer medial surface, an outer lateral surface and an intercondylar notch between first and second condyles, the intercondylar notch located between the outer medial and lateral surfaces of the femoral component;
a femoral component holder including first and second members, the first member including a base and an intercondylar post extending from the base, the intercondylar post engageable with the intercondylar notch of the femoral knee component, the second member including an extension with an engagement feature engageable with only one of the outer medial or lateral surfaces of the femoral knee component, the first and second members defining a clamp movable between a closed clamp position securing the femoral knee component and an open clamp position releasing the femoral knee component, wherein, when the clamp is in the closed clamp position, only one of the first or second condyles of the femoral component is clamped between the post and the engagement feature.

30. The surgical apparatus of claim 29, wherein the first and second members are substantially adjacent in the closed clamp position, and the first and second members are parallel and spaced apart in the open clamp position and wherein the first and second members move from the closed clamp position to the open clamp position by moving in a direction substantially perpendicular to a longitudinal axis of the first member.

31. The surgical apparatus of claim 30, further comprising first and second of opposing leaf springs coupled to the first and second members and biasing the clamp in the open clamp position.

32. The surgical apparatus of claim 30, further comprising an actuator operable to move the second member parallel and spaced apart from the first member in the open clamp position.

33. The surgical apparatus of claim 32, wherein the actuator includes:
a lever having first and second ends, the lever pivotably coupled to the first member at an intermediate portion between the first and second ends;
a shaft attached to the second member and passing through openings in first member and the first end of the lever; and
a spring coupling the second end of the lever and the first member.

34. The surgical apparatus of claim 33, further comprising a locking device rotatably coupled to the first member and including grooves, the grooves operable for capturing the lever and locking the actuator in the closed clamp position.

35. The surgical apparatus of claim 34, wherein the locking device includes an impaction surface.

36. The surgical apparatus of claim 29, wherein the engagement feature is hook-shaped and engageable to a slot of the corresponding medial or lateral surface of the femoral knee component.

37. The surgical apparatus of claim 29, wherein the engagement feature is substantially perpendicular to the intercondylar post.

38. A holder for a prosthetic femoral knee component including an outer medial surface, an outer lateral surface and an intercondylar notch between first and second condyles, the holder comprising:
a first member, the first member including a base and an intercondylar post extending from the base, the intercondylar post engageable with the intercondylar notch of the femoral knee component, the intercondylar notch located between the outer lateral and the medial surfaces; and
a second member including an extension with an engagement feature engageable with only one of the outer medial or lateral surfaces of the femoral knee component,
wherein the first and second members define a clamp movable between a closed clamp position securing the femoral knee component and an open clamp position releasing the femoral knee component, wherein, when the clamp is in the closed position, only one of the first or second condyles of the femoral component is clamped between the post and the engagement feature.

39. The holder of claim 38, wherein the engagement feature is hook-shaped and engageable to a slot of the corresponding medial or lateral surface of the femoral knee component.

40. The surgical apparatus of claim 38, wherein the first and second members are substantially adjacent in the closed clamp position, and the first and second members are parallel and spaced apart in the open clamp position, and wherein the first and second members move from the closed clamp position to the open clamp Position by moving in a direction substantially perpendicular to a longitudinal axis of the first member.

41. The surgical apparatus of claim 38, further comprising first and second of opposing leaf springs coupled to the first and second members and biasing the clamp in the open clamp position.

42. The surgical apparatus of claim 38, further comprising an actuator operable to move the second member parallel and spaced apart from the first member in the open clamp position.

43. The surgical apparatus of claim 42, wherein the actuator includes:
a lever having first and second ends, the lever pivotably coupled to the first member at an intermediate portion between the first and second ends;
a shaft attached to the second member and passing through openings in first member and the first end of the lever; and
a spring coupling the second end of the lever and the first member.

44. The surgical apparatus of claim 43, further comprising a locking device rotatably coupled to the first member and including grooves operable for capturing the lever and locking the actuator in the closed clamp position.

45. The surgical apparatus of claim 44, wherein the locking device includes an impaction surface.

46. The surgical apparatus of claim 39, wherein the engagement feature is substantially perpendicular to the intercondylar post.

47-52. (canceled)

53. The method of claim 15, wherein gripping the intercondylar notch of the femoral knee component with the post of the inserter includes inserting the post into the intercondylar notch.

54. The method of claim 15, further comprising biasing the post and the engagement feature to an open clamp position, and wherein clamping only one of the first or second condyles of the femoral knee component between the intercondylar notch and the gripped outer lateral or medial surface between the post and the engagement feature includes moving the post and engagement feature to a closed clamp position against bias.

55. The method of claim 15, wherein clamping further includes moving a first member of the inserter perpendicularly to a longitudinal axis of the first member to a position parallel to and spaced apart from a second member, the first member including the post and the second member including the engagement feature.

56. The method of claim 56, further comprising rotating a locking device to lock the first and second members relative to one another.
